(19) United States
(12) Patent Application Publication
(10) Pub. No.: US 2025/0074850 A1
(43) Pub. Date: Mar. 6, 2025

(54) METHOD AND SYSTEM FOR METHANOL PRODUCTION

(71) Applicants: Boris Davidoff, Old Westbury, NY (US); Hendrikus Johannes VAN SONSBEEK, AR Sloten (NL)

(72) Inventors: Boris Davidoff, Old Westbury, NY (US); Hendrikus Johannes VAN SONSBEEK, AR Sloten (NL)

(21) Appl. No.: 18/826,127

(22) Filed: Sep. 5, 2024

Related U.S. Application Data
(60) Provisional application No. 63/536,570, filed on Sep. 5, 2023.

(51) Int. Cl. C07C 29/157; B01D 53/22; B01D 69/02; B01D 69/08; B01J 19/08; B01J 23/42

(52) U.S. Cl. CPC C07C 29/157; B01D 53/22; B01D 69/02; B01D 69/08; B01J 19/088; B01J 23/42; B01D 2053/224; B01D 2257/504; B01D 2325/38; B01J 2219/0854; B01J 2219/0869; B01J 2219/0884; B01J 2219/0892; C07C 2523/42

(57) ABSTRACT

A method of producing methanol using a membrane gas absorption unit and at least one reactor. To produce methanol, a membrane gas absorption unit and a reactor are provided, each membrane gas absorption unit having a plurality of membranes, and each reactor having at least one section having a flocculator producing a pulsated magnetic field. The membrane gas absorption unit is first used to capture CO2 from a desired source. The captured CO2 is then supplied in a liquid form to the reactor. A hydrogen is supplied to the same reactor, and both captured CO2 and the hydrogen are then subjected to the pulsated magnetic field within the reactor to obtain a mixture of water and methanol. Finally, the water is distilled from the methanol to obtain pure methanol.

### CROSS-REFERENCE TO RELATED APPLICATIONS

This application claims all rights of priority to U.S. Provisional Patent Application No. 63/536,570, filed on Sep. 5, 2023 (pending).

### FIELD OF INVENTION

The invention relates to the field of methanol and methane production.

### BACKGROUND

In the current state of the art, methanol and methane, as a by-product, can be produced using captured CO2 that reacts with hydrogen. This gas reaction takes place at a high temperature, typically 210-270° C., under high pressure, typically in the range of 5-10 MPa, and in the presence of a catalyst, typically platinum. Further, because water is a reactional by-product of this process, after the reaction is completed, this water has to be removed via distillation. Accordingly, these reactions require a significant amount of energy. Further, production of hydrogen necessary for these reactions (even green hydrogen) also requires a large amount of energy. Thus, there is a need in the art for a less energy consuming way of producing methanol and methane.

### SUMMARY OF THE INVENTION

In accordance with the preferred embodiment of the present invention, methanol is produced using a magnetic field. Specifically, the invention is a method and system of producing methanol using a membrane gas absorption unit and at least one reactor. To produce methanol, a membrane gas absorption unit and a reactor are provided, each membrane gas absorption unit having a plurality of membranes, and each reactor having at least one section having a flocculator producing a pulsated magnetic field. The membrane gas absorption unit is first used to capture CO2 from a desired source. The captured CO2 is then supplied in a liquid form to the reactor. A hydrogen is supplied to the same reactor, and both captured CO2 and the hydrogen are then subjected to the pulsated magnetic field within the reactor to obtain a mixture of water and methanol. Finally, the water is distilled from the methanol to obtain pure methanol.

It should be understood by a person skilled in the art that the disclosed chemical reaction mechanism is not limited to the production of methanol. It can also be used in the whole chemical industry to reduce the amount of energy required to execute chemical reactions in an aqueous solution (for example: kerosine production, diesel, plastics, etc.).

### BRIEF DESCRIPTION OF THE DRAWINGS

The invention is illustrated by way of examples which are not a limitation, and the figures of the accompanying drawings in which references denote corresponding parts, and in which:

FIG. 1 shows a schematic diagram of an apparatus for methanol production in accordance with the preferred embodiment of the present invention.

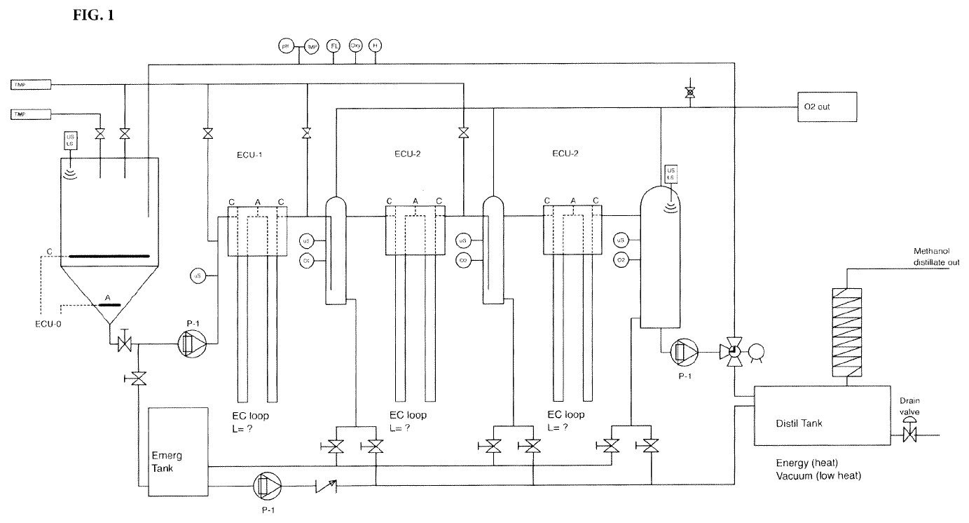

### DETAILED DESCRIPTION

In accordance with the preferred embodiment, one of the ingredients for producing the methanol and, as a by-product, methane, is CO2. The CO2 required for the reaction can be obtained by separating it from biological anaerobic processes like digesters, whiskey production or anaerobic water treatment, off gasses from burning fuel; or by capturing it from the air. Using a biogas combined with captured CO2 from the air does not require cleaning of the CO2. Therefore, this method is preferential over using the off gasses.

Capturing CO2 from the air (or another source) includes the following 3 steps:

Via hydrophobic hollow fibber membranes, CO2 will be dissolved into a KOH Solution (pH 13-14). The CO2 reacts directly with water to CO32- and 2 H3O+. The H3O+ reacts with OH— to 2H2O. So, the pH drops as a result of the reaction. At pH 11-10, HCO3- will also be formed. This step is called Membrane Gas Absorption (MGA)

The KOH solution is recycled over the MGA membranes until the pH is dropped to pH 9. At this stage most captured CO2 is transferred to HCO3-.

Via electrodialysis the HCO3- and CO32- will be transferred to CO2 and OH—. The CO2 leaves the system as a 97-98% pure CO2. The water containing the OH— can then be re-used to capture CO2.

Because the OH— is recovered, no chemicals are required to capture the CO2. Some water will evaporate through the membranes and will have to be added in time. The 3% water in the CO2 can easily be removed during liquefaction.

Capturing the CO2 in the MGA unit does not require much electricity. Recirculation at a flow rate of 0.3 m/s is enough.

However, the regeneration of the captured CO2 requires electrical energy. Based on the produced shape CO32- or HCO3- per molecule CO2 2 or 1 electron has to be supplied by the rectifier.

In accordance with the preferred embodiment, a pulsated magnetic field is introduced to speed up the reactions. Pulsated magnetic field using a very low energy is used to initiate/run chemical reactions like the production of methanol in an aqueous solution.

The following specific designs and parameters were obtained as a result of the empirical studies:

Using a pilot plant at 600 V @ 0.8 A DC at a frequency of 4 HZ for one hour to test the effect of an magnetic field on a reaction, the following was concluded.

The designed reactor/flocculator produces a pulsated magnetic field.

The pulsated magnetic field speeds up the reaction rate with an average of 1.44 in 48 minutes starting with a factor of 4.

A pulsating magnetic field will not change the chemical equilibrium state of the produced reaction. In other words, the equilibrium state based on the dosed chemicals will be reached sooner.

Based on Faraday's law the reaction rate will not be infected by electrical chemical reactions, because the current is too low to produce ions, while the conductivity is very high.

The magnetic field will maintain in the water, even after leaving it for 131 sec.

Based on the above, it is very likely that, due to the consistent magnetic field chain, reactions will take place, especially because energy will not be lost.

As known in the art, all elements are built up by protons, neutrons and electrons. Molecules are bounded elements. Thus, every element/molecule can be influenced/change its physical state using a proper frequency of a magnetic field.

The electrons in every element have their own specific orbitals and chaired in molecules, the frequency and required voltage producing the pulsated magnetic field to influence its bonding/physical state is unique.

Other reactions like the production of methanol out of CO2 in an aqueous solution can also be executed (accelerated) using a pulsating magnetic field.

Hydrogen bridges between the water molecules are responsible for the special physical behaviour of water and its ability to dissolve ions. It is clear that the bridges also contribute to the maintaining of the measured magnetic field of the water during the test.

The production of the pulsating magnetic field requires almost no energy. Although the tension is very high (600 V DC) the current is very low 0.8 A while the conductivity is very high (>50 mS/cm). Total power requirement during the test was 48 W.

This means that with a low energy reactor set-up combined with the right magnetic field frequency, products like methanol can be produced on a much more cost-effective way compared to state of the art methods which utilize big energy consuming installations.

Dissolving CO2 in a caustic environment forming carbonate combined with the pulsated magnetic field reaction will result in a very effective and cheap way to produce methanol, methane or other bio-based fuels.

### General Concept of the Invention

As explained above, all elements comprise protons, neutrons and electrons. Molecules are bounded elements. Every element/molecule can be induced to change its physical state using the proper frequency of a magnetic field.

The electrons in every element have their own specific orbitals and chaired in molecules, the frequency and required voltage producing the pulsated magnetic field to influence its bonding/physical state is unique.

In accordance with the invention, the process of production of methanol out of CO2 in an aqueous solution is executed (accelerated) using a pulsating magnetic field.

K2CO3 or KHCO3 at a concentration of 4 moles are preferably used as the CO2 source. Every other source is possible to be used with the present invention and is not limited for this disclosure.

Hydrogen bridges between the water molecules are responsible for the special physical behaviour of water and its ability to dissolve ions. It is clear that the bridges also contribute to maintain the magnetic field of the water.

Because energy will never be lost, a mercury coil will be used in the center of the flocculator reactor maintaining the measured magnetic field.

The pulsating magnetic field is produced by applying a high to very high pulsating voltage (600-10.000 V at a frequency of 0-10 HZ) to an anode while maintaining the amperage at a very low value (<1).

To avoid electrical chemical reactions the surface of the anode (negative) is kept small, while the distance to the cathode is kept long. For this reason, the inventors developed so-called flocculators or pipe reactors where the distance between the anode and cathode is long enough to ensure that the amperage is maintained under 1 A. The mercury coil is located in the center of the flocculator so as to maintain the magnetic field.

Several flocculators at different magnetic field strengths can be used, if it is necessary to dose basic chemicals to the flocculator at different injection points. Note that the frequency of all the flocculators in the system has to be the same, and equal qua timing.

At the end of each flocculator a small buffer tank is introduced to remove reaction products. In the methanol production process, this is used to remove oxygen.

Peroxide (H2O2) or OH radicals can be used to as a hydrogen source. Platinum plates are preferably used as catalysts because free hydrogen molecules will be on their surfaces. Accordingly, every flocculator in accordance with the present invention includes platinum bars positioned after the peroxide dosing points. The length and number of the platinum bars will depend on the number and length of the used flocculators. Cobalt or other suitable materials can also be used as catalysts.

### The Inventive Process Set Up

In accordance with the preferred embodiment of the invention, CO2 is captured using membranes combined with KOH at pH13-14. At the moment that the liquid is saturated, it will be pumped to the reactor.

In the reactor, the reaction HCO3- and CO32 with peroxide in a pulsated magnetic field will produce methanol and water. Also, during the reaction Hydrogen is used so the pH will rise.

Distillation is then required to remove the water from the methanol. Also, the Kalium will stay in the water fraction of the distillation column.

This water (with a high pH) can then be re-used in the MGA unit and recycled.

### The Inventive MGA Unit:

The MGA unit contains preferably the following components:

MGA membrane capturing system. In one embodiment of the invention, hollow fibre membranes are used as the membrane capturing system. Flat sheet membrane modules (design I3) can also be used to speed up the performance.

A 4-5 moles KOH solution is used to capture the CO2.

Three (3) buffer tanks are preferably used. One is in operation for capturing CO2, the second one is for capturing the released KOH solution from the distillation column. The third one is for feeding the reactor.

Recirculation system containing pH and conductivity control to recycle the liquid over the MGA membranes.

### Reactor System:

In the reactor the methanol (and methane as a by-product) will be produced out of the captured CO2 by the membrane unit. Peroxide is added to the liquid, if required acid is added to set the right pH.

The reactor preferably contains 3-7 reaction sections containing a flocculator producing a pulsated magnetic field. Based on the concentration every reactor could have its own pulsation frequency and its own rectifier. Every reactor system has its own recirculation-controlled system. Every flocculator will be equipped with platinum rings (Raschig) that are used as catalysts. The system further preferably includes a buffer tank to be able to circulate over the reactors (if required); a feed/recirculation system; a pH/conductivity measurement/control system; a feed to distillation column; and a peroxide dosing system.

The produced methanol is dissolved into a water/KOH solution. To purify the methanol, it has to be distilled out in a distillation column. Methanol/water distillation is a very common process widely used in the industry. A standard column can be used to separate the produced methanol. A column equipped with Raschig packing which is preferred due to its simplicity to operate it. To minimize energy consumption a heat pump can be evaluated into the design later.

The water/KOH solution produced by the distillation column can be pumped back to the MGA unit and re-used to capture the CO2. As an alternative membrane distillation can also be used to remove the water from the produced methanol.

The preferred embodiment of a pilot plant set-up for methanol production is described below. In this case three (3) flocculators are used, for other reactions this can be extended or released based on the reaction requirements. The set-up contains the following main equipment: a buffer tank equipped with an anode and cathode. Also, the feed solution from the MGA unit (K2CO3) and, if required, H2O2 will be added to the tank. Magnetic feed pump is used for circulation over the flocculators. The three flocculators are each equipped with its own rectifier and voltage (frequency is the same). Also, every flocculator contains its own buffer tank with gas trap, to remove produced oxygen, and pH and conductivity measurement means to follow the reaction.

The buffer tank at reactor 3 (the last one in this embodiment) is preferably bigger than the other ones because part of the aquas solution will be pumped back to the feed tank while the rest will be pumped to the distillation column to remove the water from the produced methanol.

In case of an emergency, the reactors can be drained into a provided emergency tank to be removed from the magnetic field eliminating the reactions.

Further the pH, conductivity, flow, and oxygen are preferably measured in the recycle stream.

The key parameters of the reaction are the frequency of the magnetic field, the voltage required to generate the field and the flow/electrodes located in the flocculator. Also, the shape and the amount of catalyst (platinum or cobalt, for example) will influence the yield and the reaction rate.

Other attention point is that the material of the reactor should be plastic (PE or PP) because the reactor material is not allowed to influence the magnetic field.

The electrodes can be made of stainless steel.

In the system disclosed above, methane can be formed as a by-product. Methane is not solvable in the liquid, so it will be formed as bubbles that, via a 3-phase separator, can be removed from the reactor.

In the preceding specification, the invention has been described with reference to specific exemplary embodiments thereof. It will however, be evident that various modifications and changes may be made thereto without departing from the broader spirit and scope of the invention as set forth in the claims that follow. The specification and drawings are accordingly to be regarded in an illustrative manner rather than a restrictive sense.

1. A method of producing methanol, the method comprising:
providing a membrane gas absorption unit comprising a plurality of membranes;
using said membrane gas absorption unit to capture CO2 from a desired source;
providing a reactor, each reactor comprising at least one section having a flocculator producing a pulsated magnetic field;
supplying said captured CO2 in a liquid form to said reactor;
supplying a hydrogen to said reactor;
subjecting said captured CO2 and said hydrogen to said pulsated magnetic field within said reactor to obtain a mixture of water and methanol;
distilling said water from said methanol.

2. The method of producing methanol according to claim 1, wherein said hydrogen is supplied in the form of a peroxide.

3. The method of producing methanol according to claim 1, wherein said step of using said membrane gas absorption unit to capture CO2 further comprises providing said membrane gas absorption unit with a plurality of hydrophobic hollow fiber membranes; dissolving CO2 into a KOH solution having a pH factor of pH13-14 via said hydrophobic hollow fiber membranes; recycling said KOH solution over the hydrophobic hollow fiber membranes until the pH factor is dropped to pH 9; transforming the resulting substance into CO2 via electrodialysis.

4. The method of producing methanol according to claim 3, further comprising a step of adding an acid to achieve a desired pH factor.

5. The method of producing methanol according to claim 1, further comprising a step of forming methane as a by-product, wherein said methane is formed as bubbles that.

6. The method of producing methanol according to claim 5, further comprising removing said methane from the reactor via a 3-phase separator.

7. A system for producing methanol, the system comprising:
a membrane gas absorption unit having a plurality of membranes, said membrane gas absorption unit being configured to capture CO2 from a desired source; and
at least one reactor, each reactor comprising at least one section having a flocculator producing a pulsated magnetic field, said at least one reactor being connected to said membrane gas absorption unit so as to receive captured CO2 in a liquid form from said membrane gas absorption unit,
wherein said at least one reactor is configured to receive a hydrogen and to subject said captured CO2 and said hydrogen to said pulsated magnetic field within said reactor so as to obtain a mixture of water and methanol.

8. The system for producing methanol according to claim 7, wherein said flocculator further comprises an anode, a cathode and a catalyst.

9. The system for producing methanol according to claim 8, wherein said catalyst is made of platinum.

10. The system for producing methanol according to claim 7, further comprising a buffer tank configured to circulate over the reactor; a feed recirculation system; a pH conductivity measurement and control system; a distillation column having a feeder connected to said reactor, said distillation column being configured to distil said water from said methanol; and a peroxide dosing system configured to supply peroxide to said membrane gas absorption unit.
